(19) United States
(12) Patent Application Publication
(10) Pub. No.: US 2016/0199214 A1
(43) Pub. Date: Jul. 14, 2016

(54) Collection Apparatus with Adjustable Length

(71) Applicant: Michael Koretsky, Hightstown, NJ (US)

(72) Inventor: Michael Koretsky, Hightstown, NJ (US)

(21) Appl. No.: 14/993,807

(22) Filed: Jan. 12, 2016

Related U.S. Application Data
(60) Provisional application No. 62/102,365, filed on Jan. 12, 2015.

(51) Int. Cl. A61F 5/453

(52) U.S. Cl. CPC A61F 5/453

(57) ABSTRACT

A sperm collecting apparatus that has a generally cylindrical housing and an inner sleeve disposed inside the housing and made from a gel-like material. The housing defines an interior chamber and has a proximal opening at a proximal end of the housing. The inner sleeve defines an internal insertion channel that is sized for closely receiving a human penis and has an insertion opening at the proximal opening of the housing, The length of the housing is adjustable in length. The housing includes a proximal end configured for telescopic engagement with a housing distal end so that the housing proximal and distal ends can be moved longitudinally with respect to each other to adjust the length of the housing. The housing distal portion includes two opposing side portions configured so they can be removably assembled together.

### RELATED APPLICATION

This application claims the benefit of U.S. Provisional Application No. 62/102,365, filed Jan. 12, 2015, entitled “Collection Apparatus with Adjustable Length,” which is incorporated herein in its entirety by this reference.

### BACKGROUND

The present invention relates to a sperm collecting apparatus. In particular, the present invention relates to such an apparatus that is adjustable in length.

Sperm collection devices have been previously known. Examples of such devices can be seen in U.S. Pat. Nos. 5,807,360, 7,753,895 and 7,993,315. Typically, such devices have a gel-like inner sleeve disposed within a housing and defining an insertion opening and channel for receiving a user's penis.

It is an object of the present invention to provide an improved sperm collection apparatus that is adjustable in length.

It is another object of the present invention to provide an improved sperm collection apparatus that is reusable and can be easily cleaned without removing the inner sleeve.

Additional objectives and advantages of the invention will be set forth in the description that follows, and in part will be apparent from the description, or may be learned by practice of the invention. The objects and advantages of the invention may be realized and obtained by means of the instrumentalities and combinations pointed out in this specification.

### SUMMARY

To achieve the foregoing objectives, and in accordance with the purposes of the invention as embodied and broadly described in this document, there is provided a sperm collecting apparatus that has a generally cylindrical housing and an inner sleeve disposed inside the housing and made from a gel-like material. The housing defines an interior chamber and has a proximal opening at a proximal end of the housing and a removable cap for covering the opening of the housing proximal end. The inner sleeve defines an internal insertion channel that is sized for closely receiving a human penis and has an insertion opening at the proximal opening of the housing. The length of the housing is adjustable so that it can accommodate penises of different lengths. The housing distal portion can have two opposing side portions configured so they can be removably assembled together.

In one embodiment, the housing proximal end is configured for telescopic engagement with a housing distal end so that the housing proximal and distal ends can be moved longitudinally with respect to each other to adjust the length of the housing. For example, the housing can have an intermediate cylindrical portion and the distal end can be disposed telescopically over the intermediate portion. A spiral groove can be disposed on an exterior surface of the housing intermediate portion and the housing distal portion can include one or more inward projections configured for engaging the spiral groove. A plurality of pockets can be disposed along the length of the spiral groove, and each of the inward projections can be sized and shaped to fit within each of the pockets. In this configuration, as the housing distal end is rotated by the user, each of the engaging inward projections moves incrementally from one pocket to the next along the path of the corresponding spiral groove and causes the housing distal end to telescopically move along the intermediate cylindrical portion away from the housing proximal end, thereby extending the length of the housing.

### BRIEF DESCRIPTION OF THE DRAWINGS

The accompanying drawings, which are incorporated in and constitute a part of the specification, illustrate the presently preferred embodiments and methods of the invention and, together with the general description given above and the detailed description of the preferred embodiments and methods given below, serve to explain the principles of the invention.

FIG. 1 is a side elevation view of one exemplary embodiment of a collection apparatus according to the present invention, showing the apparatus in the normal or non-extended position with the removable cap on.

FIG. 2 is a side elevation view of the embodiment of FIG. 1, showing the apparatus in the normal or non-extended position with the cap removed.

FIG. 3 is a side elevation view of the embodiment of FIG. 1, showing the apparatus in the extended position with the cap on.

FIG. 4 is a side elevation view of the embodiment of FIG. 1, showing the apparatus in the extended position with the cap removed.

FIG. 5 is a cross-sectional elevation view of one exemplary embodiment of the inner sleeve of the collection apparatus.

FIG. 6 is a cross-sectional elevation view of another exemplary embodiment of the sleeve of the collection apparatus.

FIG. 7 is a perspective view of the collection apparatus of FIG. 1 showing cap removed and the shell halves of the housing distal end disassembled.

FIG. 8 shows a part of the interior of the housing distal portion.

FIG. 9 is an exploded perspective view of the collection apparatus of FIG.

FIGS. 10A-10D show how the collection apparatus of FIG. 1 can be lengthened by a rotating or twisting action and disassembled for cleaning.

### DESCRIPTION

Reference in this application is made to presently preferred embodiments of the invention. While the invention is described more fully with reference to these examples, the invention in its broader aspects is not limited to the specific details, representative devices, and illustrative examples shown and described. Rather, the description is to be understood as a broad, teaching disclosure directed to persons of ordinary skill in the appropriate arts, and not as limiting upon the invention.

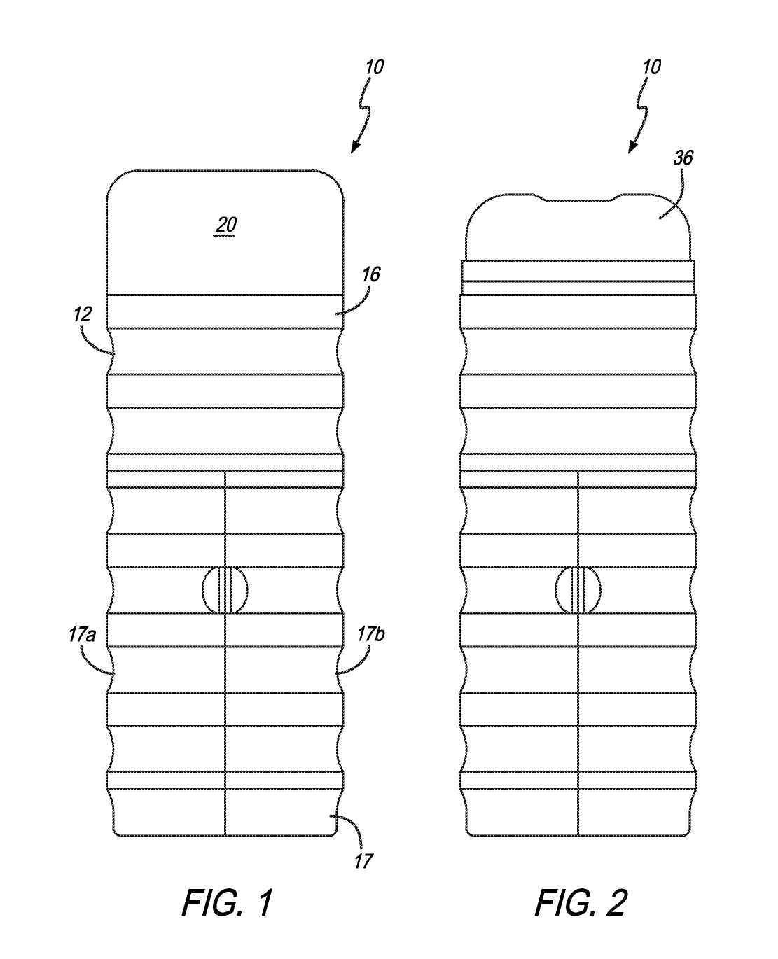

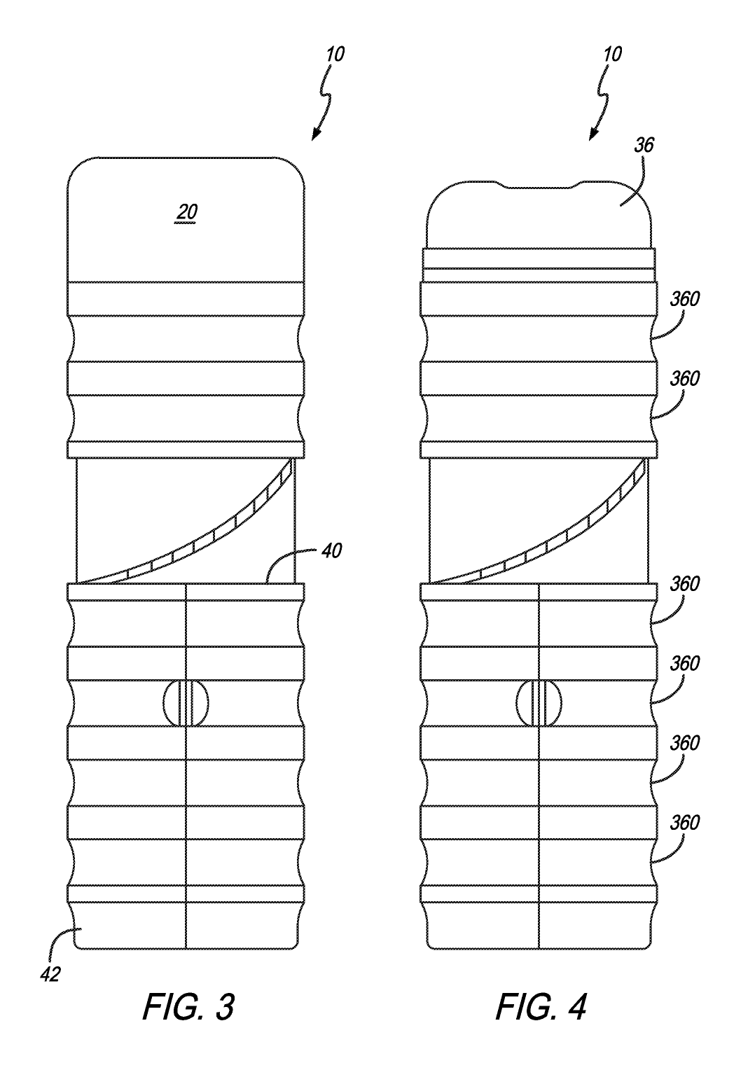

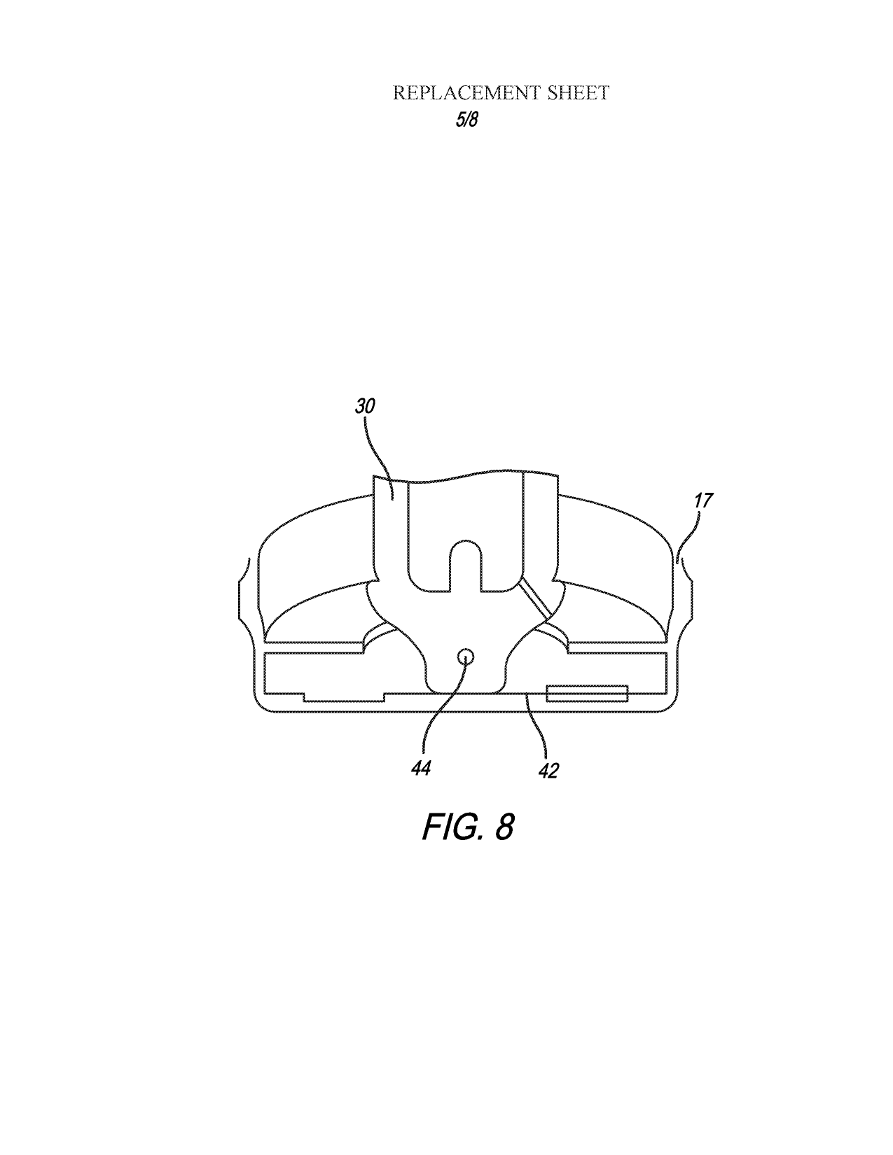

Referring generally to FIGS. 1-10, a presently preferred embodiment of a sperm collection apparatus 10 according to the invention includes a container having a generally cylindrical housing 12 defining an interior chamber 14 and having a proximal portion or end 16 and a distal portion or end 17. The housing proximal end 16 has an opening 18 and is adapted for attaching a removable cap 20 to cover the opening 18. As shown in the figures, the housing 12 preferably has a series of peripheral grooves 360 along a majority of its length to provide for better gripping of the housing during use.

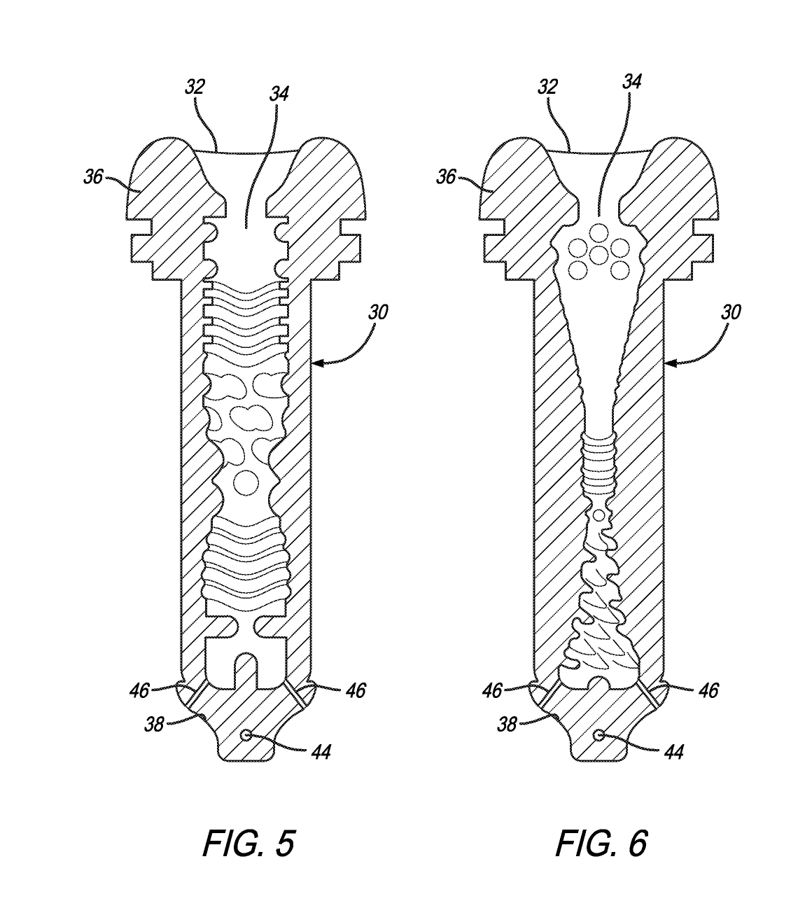

An inner sleeve 30 is mounted to the housing proximal end 16 and is disposed inside the housing 12. The inner sleeve 30 is made from a stretchable gel-like material, as is known in the art, and defines an internal elongated insertion channel 34 (as can be seen in FIGS. 5 and 6) that is sized for closely receiving a user's penis. The inner sleeve 30 has a proximal end 36 and a distal end 38. Near the housing proximal opening 18, the inner sleeve 30 defines an insertion opening 32 that is in communication with the insertion channel 34, as can be seen in FIGS. 5 and 6. The insertion opening 32 is exposed when the removable cap 20 is removed. The insertion channel 34 can be configured with varying diameters, geometries, projections, ridges and folds along its length to provide a user with the sensation of a life-like canal and with enhanced sensations during use. The inner sleeve proximal end 36 is mounted to a mounting ring 19 that is mounted to the housing proximal opening 18. A security ring 21 holds and locks the inner sleeve proximal end 36 in place on the housing proximal end 16.

The housing distal end 17 is formed by two shell halves 17a, 17b that can be assembled to form the housing distal end 17 as described in more detail below. The housing 12 includes an intermediate cylindrical section 22 that fits telescopically within the housing distal end 17 so that the housing distal end 17 can be moved longitudinally to adjust the length of the housing 12 as described in more detail below. The shell half 17a has a set of tabs 27a that clip into corresponding slots 28b on the other shell half 17b so that the shell halves 17a, 17b can be assembled to form a cylindrical housing distal end 17 having an open end 40 and a closed end 42. Similarly, the shell half 17b has a set of tabs that clip into corresponding slots on the shell half 17a.

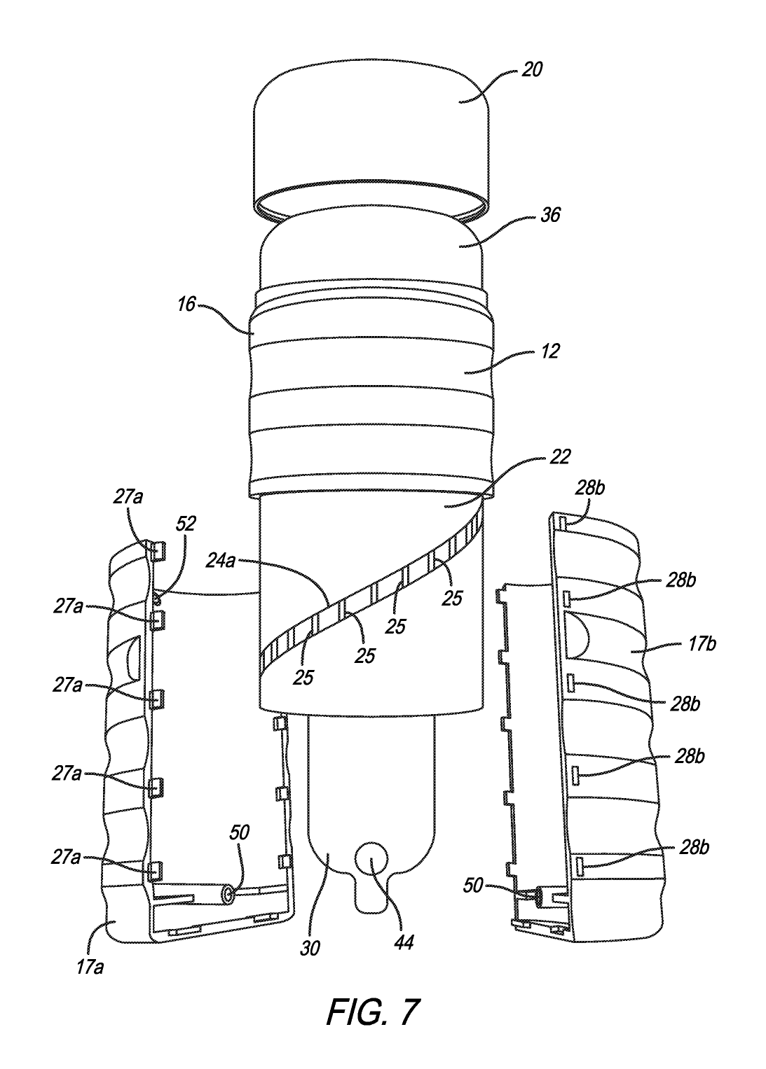

As can be seen in FIGS. 7-9, opposing spiral grooves 24 are formed in the exterior surface of the intermediate cylindrical section 22 of the housing 12. The spiral grooves 24 include transverse ridges 25 to form pockets along the length of the spiral grooves 24. An inward projection 52 is disposed on the internal surface of each of the shell halves near the open end 40a, 40b for engaging the corresponding spiral groove 24a, 24b of the intermediate cylindrical section 22 when the shell halves 17a, 17b are assembled over the intermediate cylindrical section 22. Each of the inward projections is sized and shaped to fit within one of the pockets formed in the spiral groove 24. In this configuration, as the housing distal end 17 is rotated in a counterclockwise direction by the user, each of the engaging inward projections moves incrementally from one pocket to the next along the path of the corresponding spiral groove 24 and causes the housing distal end 17 to telescopically move along the intermediate cylindrical section 22 away from the housing proximal end 16, thereby extending the length of the housing 12.

Still referring to FIGS. 7-9, the inner sleeve distal end 38 includes a transverse hole 44 for attaching the inner sleeve distal end 38 to the housing distal end 17. Each of the shell halves 17a, 17b includes a cylindrical projection 50 that can be inserted into the inner sleeve transverse hole 44. During assembly of the housing distal end 17, the user inserts one of the cylindrical projections through the hole 44 before clipping the shell halves 17a, 17b together. When assembled in this configuration, the inner sleeve distal end 38 remains securely attached to housing distal end 17. As the length of the assembled unit 10 is increased by twisting the housing distal end 17, the inner sleeve 30 stretches by a corresponding length.

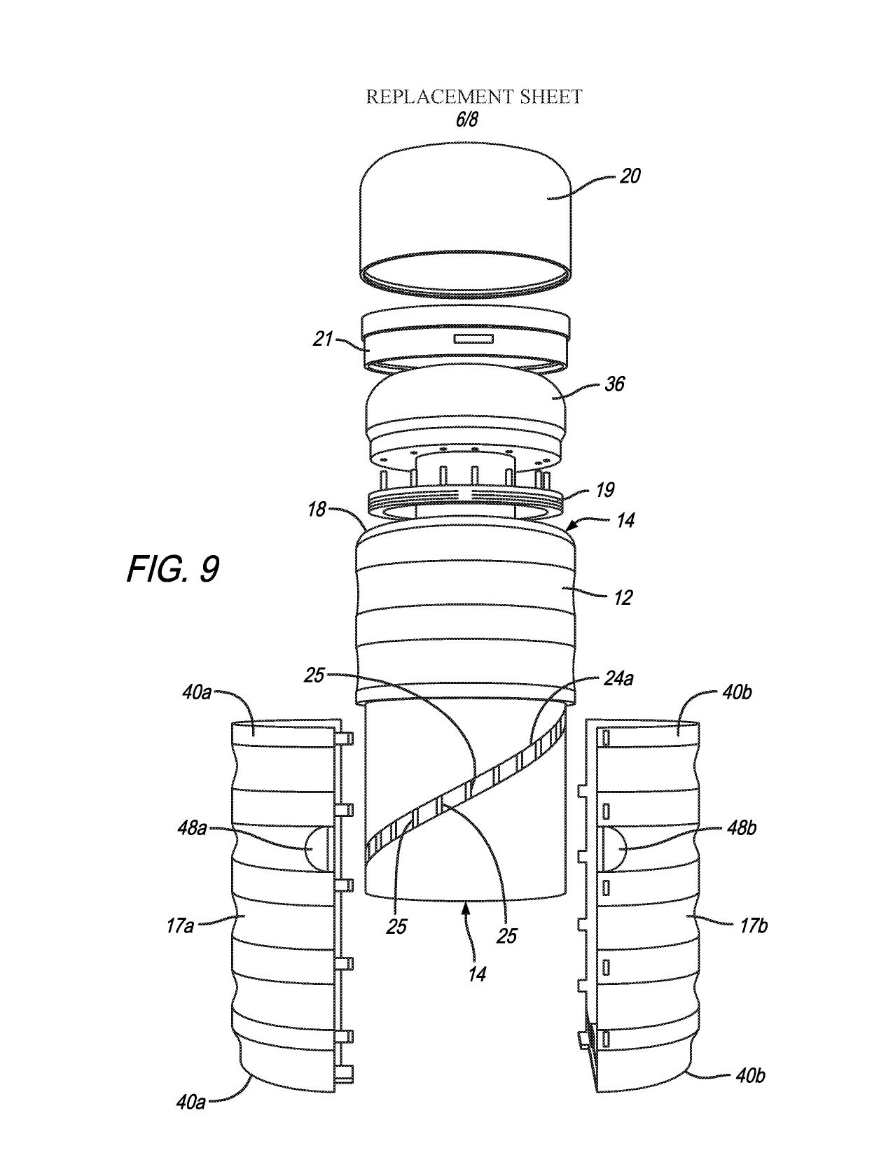

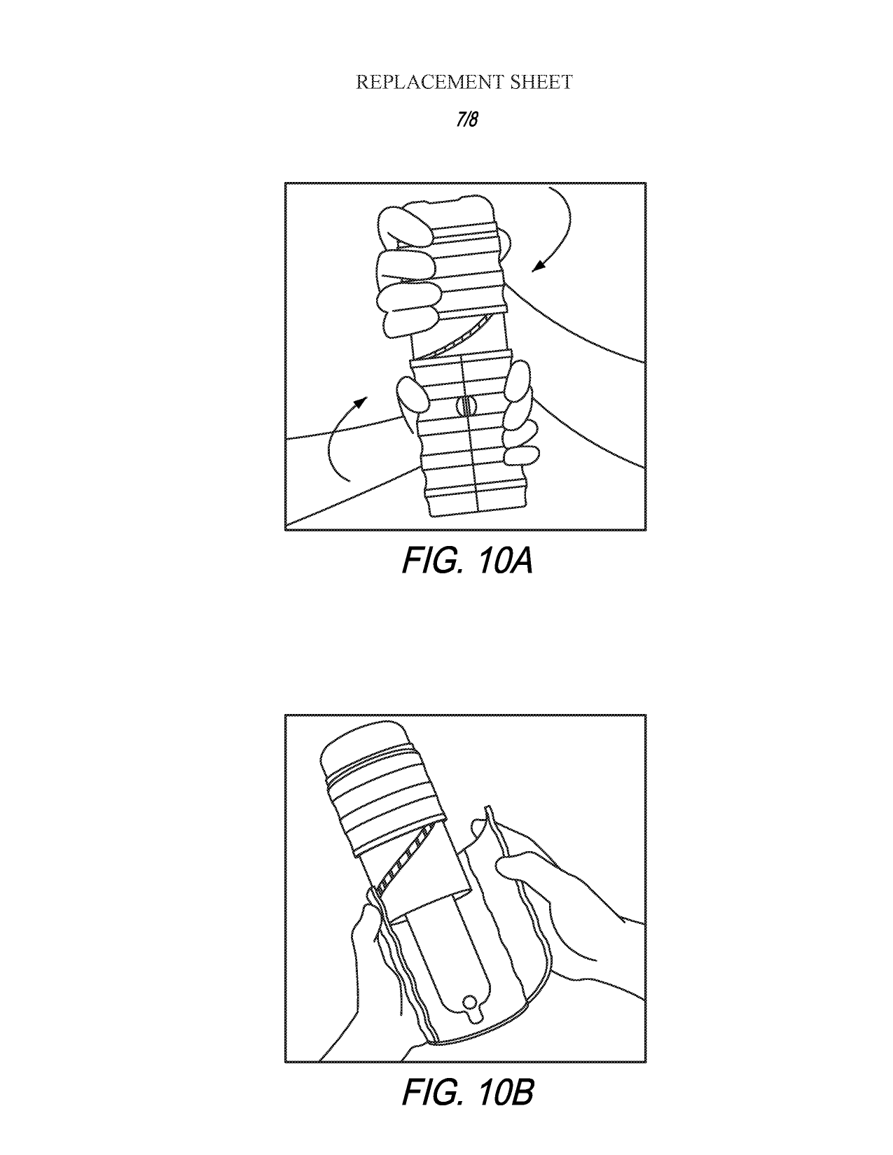

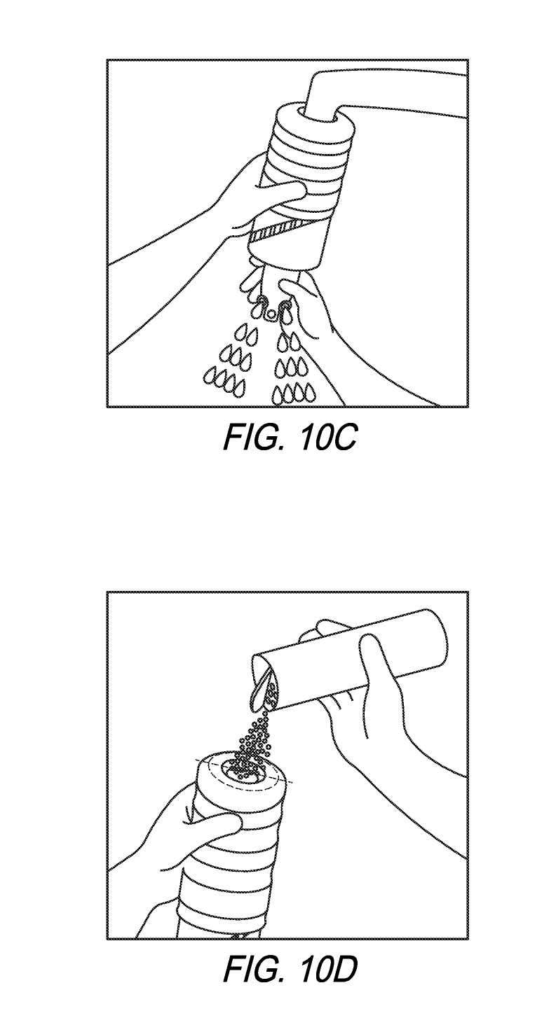

As shown in FIGS. 10A-10B, after use, the apparatus 10 can be cleaned by disassembling and removing the shell halves 17a, 17b of the housing distal end 17 and then rinsing out the inner sleeve 30 with warm water or another suitable cleaning fluid. To facilitate this cleaning process, the inner sleeve 30 includes draining holes 46 in its distal end 38 (see FIG, 8). To disassemble the distal end 17, the housing distal end 17 is moved to its fully extended position (FIG. 10A) and the shell halves 17a, 17b are unclipped from each other (FIG. 10B). As shown in FIGS. 7 and 9, depressions 48a, 48b are provided in the outer surface of the shell halves 17a, 17b to facilitate gripping of the shell halves 17a, 17b to disassemble them. The inner sleeve 30 can be allowed to dry naturally, Also, powder can be introduced into the inner sleeve 30 to help completely dry/absorb any small amounts of remaining liquid (FIG. 10D).

Having read this disclosure, it will also be understood by those having skill in the art that modifications may be made to the invention without departing from its spirit and scope. Therefore, the invention in its broader aspects is not limited to the specific details, representative devices, and illustrative examples shown and described. Accordingly, departures may be made from such details without departing from the spirit or scope of the general inventive concept.

What is claimed is:

1. A sperm collection apparatus comprising:
a housing defining an interior chamber and having a proximal opening at a proximal end of the housing, wherein the proximal opening is in communication with the interior chamber; and
an inner sleeve comprising a gel-like material and disposed within the housing, wherein the inner sleeve defines an internal insertion channel that is sized for closely receiving a human penis and has an insertion opening at the proximal opening of the housing;
wherein the length of the housing is adjustable.

2. The collection apparatus of claim 1 wherein the housing proximal end is configured for telescopic engagement with a housing distal end and wherein the housing proximal and distal ends can he moved longitudinally with respect to each other to adjust the length of the housing.

3. The collection apparatus of claim 1 wherein the housing includes an intermediate portion and a distal portion disposed telescopically over the intermediate portion.

4. The collection apparatus of claim 3 further comprising a spiral groove disposed on an exterior surface of the housing intermediate portion and the housing distal portion includes an inward projection adapted for engaging the spiral groove.

5. The collection apparatus of claim 4 wherein the spiral groove includes a plurality of pockets disposed along the length of the spiral groove and the inward projection is sized and shaped to fit within one of the pockets.

6. The collection apparatus of claim 2 wherein the housing distal end comprises two opposing side portions configured so they can be removably assembled together.

7. A sperm collection apparatus comprising:
a housing defining an interior chamber and having a proximal end and a distal end, wherein the proximal end of the housing defines a housing proximal opening that is in communication with the interior chamber; and
a stretchable inner sleeve having a proximal end and a distal end and defining an internal insertion channel that is sized for closely receiving a human penis, wherein the insertion channel has an insertion opening disposed at the proximal end of the sleeve;
wherein the inner sleeve is configured to be disposed within the housing with the inner sleeve proximal end mounted to the housing proximal end and the inner sleeve distal end mounted to the housing distal end;
wherein the housing proximal end and distal end are configured for telescopic engagement with each other so that they can be moved longitudinally to adjust the length of the housing.

8. The collection apparatus of claim 7 further comprising a mounting ring mounted to the housing proximal end, wherein the inner sleeve is mounted to the mounting ring.

9. The collection apparatus of claim 7 wherein a security ring is configured to hold the inner sleeve proximal end to the housing proximal end.

10. The collection apparatus of claim 7 further comprising a spiral groove disposed on a surface of one end of the housing and a projection on the other end of the housing adapted for engaging the spiral groove.

11. The collection apparatus of claim 10 wherein the spiral groove includes a plurality of pockets disposed along the length of the spiral groove and the projection is sized and shaped to fit within one of the pockets.

12. The collection apparatus of claim 7 wherein the housing distal end comprises two opposing side portions configured so they can be removably assembled together.

13. The collection apparatus of claim 12 wherein each of the opposing side portions includes a tab that engages a corresponding slot on the other opposing side portion.

14. A sperm collection apparatus comprising:
a housing defining an interior chamber and having a proximal opening at a proximal end of the housing, wherein the housing proximal opening is in communication with the interior chamber; and
a stretchable inner sleeve disposed within the housing and defining an internal insertion channel that is sized for closely receiving a human penis through an insertion opening at the proximal opening of the housing;
wherein the housing includes an intermediate portion and a distal portion disposed telescopically over the intermediate portion; and
wherein a spiral groove is disposed on a surface of the housing intermediate portion and the housing distal portion has a projection configured for engaging the spiral groove.

15. The collection apparatus of claim 14 further comprising a mounting ring mounted to the housing proximal end, wherein the inner sleeve is mounted to the mounting ring.

16. The collection apparatus of claim 14 wherein a security ring is configured to hold the inner sleeve to the housing proximal end.

17. The collection apparatus of claim 14 wherein the spiral groove includes a plurality of pockets disposed along the length of the spiral groove and the projection is sized and shaped to fit within one of the pockets.

18. The collection apparatus of claim 14 wherein the housing distal portion comprises two opposing side portions configured so they can be removably assembled together.

19. The collection apparatus of claim 18 wherein each of the opposing side portions includes a tab that engages a corresponding slot on the other opposing side portion.
